(19) United States
(12) Patent Application Publication
(10) Pub. No.: US 2024/0269004 A1
(43) Pub. Date: Aug. 15, 2024

(54) METHOD FOR PROVIDING CONTROL DATA FOR AN OPHTHALMOLOGICAL LASER

(71) Applicant: SCHWIND EYE-TECH-SOLUTIONS GMBH, Kleinostheim (DE)

(72) Inventors: Samuel ARBA MOSQUERA, Aschaffenburg (DE); Shwetabh VERMA, Aschaffenburg (DE)

(21) Appl. No.: 18/433,839

(22) Filed: Feb. 6, 2024

(30) Foreign Application Priority Data
Feb. 9, 2023 (DE) 10 2023 103 190.2

(51) Int. Cl. A61F 9/008; G16H 20/40

(52) U.S. Cl. CPC A61F 9/00806; A61F 9/00829; G16H 20/40; A61F 2009/00872; A61F 2009/00878

(57) ABSTRACT

A method for providing control data for an ophthalmological laser of a treatment apparatus is disclosed. The method provides the steps of: ascertaining visual disorder correction data for correcting a cornea of an eye; determining Zernike polynomials from the ascertained visual disorder correction data; ascertaining an offset vector from a pupil center to a further preset reference center of the eye; calculating corrected Zernike polynomials, in which higher order aberrations are calculated by means of an adaptation of the corresponding Zernike polynomials by the offset vector; and providing the control data for the treatment apparatus, wherein the control data is generated by means of the corrected Zernike polynomials.

### FIELD

The invention relates to a method for providing control data for an ophthalmological laser of a treatment apparatus, to a control device, which is configured to perform the method, and to a treatment apparatus with such a control device. Furthermore, the invention relates to a computer program including commands, which cause the treatment apparatus to execute the method, and to a computer-readable medium, on which the computer program is stored.

### BACKGROUND

Treatment apparatuses and methods for controlling lasers for correcting an optical visual disorder of a cornea are known in the prior art. Therein, a pulsed laser and a beam focusing device can for example be formed such that laser beam pulses effect a photodisruption or ablation in a focus situated within the tissue of the cornea to separate a lenticule from the cornea for correcting the cornea.

For planning the correction, correction profiles are usually determined with the aid of preceding diagnostic measurements, by which visual disorder correction data can be determined. For example, a corneal topography can be established, wherein the correction profile, that is the volume body to be removed, is adjusted based on the topography. Alternatively, the correction profile can for example be planned with the aid of wavefront measurements, wherein here, since the wavefronts are measured through the pupil, the pupil center serves as a reference for adjusting the correction profile. In case of an asymmetric or decentered cornea, there is disagreement, if the correction profile is to be adjusted to a pupil center or a corneal vertex. Therein, an incorrectly placed correction profile can result in an under-correction and other undesired side effects, such as for example higher order aberrations. Alternatively, visual disorder correction data can for example be ascertained by means of a subjective refraction of a glasses correction or a glasses adjustment. Herein, different lenses are successively held against a patient to be examined by means of a phoropter, wherein the correction is determined from the subjective perception of the patient. Thus, low order aberrations can in particular also be ascertained, which can for example be expressed by means of Zernike polynomials.

The human eye is an optical system, which is substantially composed of four non-coaxial optical elements. Although the optical surfaces are nearly coaxially aligned, the deviations from a perfect optical alignment result in a series of optical and neuronal axes and the interrelations thereof. Besides the optical axes, a target is sharpest seen if it is in one line with the fixation target and the fovea of the retina, the so-called visual axis. Due to the discrepancy between the optical center (projection of the optical axis to the retina) and the visual center (which corresponds to the fovea), there is disagreement if a correction strategy of the cornea is to be adapted to maximize the quality of a wavefront on the fovea or to improve the optical system as a whole.

### SUMMARY

Therefore, it is the object of the invention to provide control data for correcting a cornea, in which coupling effects due to non-coaxially situated components of the eye are taken into account.

This object is solved by the method according to the invention, the devices according to the invention, the computer program according to the invention as well as the computer-readable medium according to the invention. Advantageous embodiments of the invention are disclosed in the respective dependent claims, the following description as well as the figures.

The invention is based on the idea that Zernike polynomials, by means of which higher order aberrations can be described, are adapted corresponding to an offset from reference centers, at which the correction can be planned.

By the invention, a method for providing control data for an ophthalmological laser of a treatment apparatus is provided for correcting a cornea, in particular an asymmetric and/or decentered cornea. This means that the method can preferably be applied for a cornea, in which an asymmetry of the corneal vertex with the pupil center is present, which means that they are not on a visual or optical axis. The method, which can be performed by a control device, includes, as steps, ascertaining visual disorder correction data for correcting a cornea of an eye, determining Zernike polynomials from the ascertained visual disorder correction data, which is adjusted based on the first reference center or a second reference center of the eye, and ascertaining an offset vector from the first reference center, in particular a pupil center, to the second reference center of the eye. Herein, the first and the second reference center can preferably differ from each other.

This means that visual disorder correction data can be ascertained, by which the cornea can be adapted for correction by means of refractive laser treatment. For example, the visual disorder correction data can include an ablation profile and/or wavefront data from an aberrometer measurement. Zernike polynomials can then be ascertained from the visual disorder correction data, which represent wavefronts, wherein the different orders of the polynomials can describe imaging errors of the eye. Therein, the Zernike polynomials are usually adjusted based on a pupil center, since the measurement of the wavefronts is performed through the pupil center. Furthermore, an offset vector can be ascertained from the first reference center, which can in particular be the pupil center, a corneal center or a corneal apex, to a further, second preset reference center of the eye, the position of which has preferably been projected into the same plane as that of the pupil center. In particular, a projection of a corneal vertex can be used to determine the offset vector from the corneal vertex to the pupil center or vice versa. Therein, a translation and/or a rotation is preferably specified by the offset vector. The second reference center can for example also be a visual axis, optical axis, achromatic axis, a corneal apex or a corneal vertex.

Furthermore, the method according to the invention includes calculating corrected Zernike polynomials, in which higher order aberrations are calculated by means of an adaptation of the corresponding Zernike polynomials by the offset vector, and providing the control data for the treatment apparatus, wherein the control data is generated by means of the corrected Zernike polynomials.

This means that the original Zernike polynomials can be adapted or shifted and/or rotated by means of the offset vector to obtain corrected Zernike polynomials. By adapting the Zernike polynomials, thus, effects of the further reference center can be taken into account, which better describes higher order aberrations for the eye. Higher order aberrations can be described by the Zernike polynomials from the second order and for example include astigmatism, defocus, trefoil and coma. In that the higher order aberrations can be adapted by means of the offset vector, a correction of the cornea can be planned in improved manner, whereby these effects can be minimized or avoided.

In other words, it can be provided to adapt the visual disorder correction data, in particular wavefront measurements, which is adjusted based on the first reference center, in particular based on the pupil center, to a further second reference center to better consider higher order aberrations. Thereto, the Zernike polynomials, which describe the higher order aberrations, can be shifted to the further second reference center, in particular a visual axis or a corneal vertex, by means of the offset vector.

Alternatively, visual disorder correction data, which is ascertained from a subjective refraction measurement (manifest refraction) or a so-called glasses correction, can be adapted. From this subjective refraction, which can in particular be adjusted to the second reference center, preferably to a corneal vertex or a visual axis, Zernike polynomials or aberrations can also be determined, but which, due to the different reference centers, cannot be directly compared to the wavefront data, which has been ascertained based on the pupil center. In order to compensate for this difference, these aberrations can thus be adapted by means of the offset vector.

This approach can also be applied for further wavefront descriptions, for example Fourier series, G-polynomials or wavelets. The control data can include a respective dataset for positioning and/or for focusing individual laser pulses in the cornea. Additionally or alternatively, a respective dataset for adjusting at least one beam device for beam guidance and/or beam shaping and/or beam deflection and/or beam focusing of a laser beam of the respective laser can be included in the control data.

By the invention, the advantage arises that improved control data for correcting the cornea can be provided since aberrations are better taken into account.

The invention also includes embodiments, by which additional advantages arise.

In an embodiment at least the Zernike polynomials for defocus, astigmatism, coma, trefoil and/or spherical aberration are adapted by means of the offset vector. Thus, the most important aberrations can be taken into account for a treatment, which results in better treatment results.

In a further embodiment the visual disorder correction data is adjusted based on the first reference center, in particular a pupil center, and the Zernike polynomials are corrected by the offset vector towards the second reference center, in particular towards a corneal vertex. In other words, the Zernike polynomials have the pupil center as the reference center, which can be determined by wavefront measurements. Herein, the further second reference center, to which the offset vector is determined, can preferably be the corneal vertex, which in particular serves as a reference for the visual axis. Thus, the Zernike polynomials can be adapted from the first reference center, in which they were measured, to the second reference center, in particular the actual visual axis, by the offset vector, which results in improved correction results.

In a further embodiment the first reference center is a visual axis of the eye, in particular a corneal vertex, and Zernike polynomials at least of the second order are determined based on a predetermined subjective refraction of a glasses correction, which is in particular ascertained by means of a phoropter measurement, wherein these Zernike polynomials are corrected by the offset vector. In other words, the visual disorder correction data includes the subjective refraction or glasses correction, which can be determined by means of a phoropter measurement. From this measurement, second order Zernike polynomials, thus astigmatism and defocus, can be ascertained, in particular independently of a wavefront measurement. Since the subjective refraction/glasses correction is ascertained based on the visual axis of the eye, which can be associated with the corneal vertex, and the occurrence of the aberrations due to the offset between the corneal vertex and the further second reference center, in particular the pupil center, is assumed, at least the second order Zernike polynomials, which have been ascertained from the subjective refraction, can thus be compensated for by means of the offset vector. Preferably, the subjective refraction can only be calculated for a certain diameter, wherein the Zernike polynomials are corrected for the same diameter. By this embodiment, the advantage arises that Zernike polynomials, which are determined from a subjective refraction and not from direct wavefront measurements, can also be adapted in improved manner. This results in improved treatment results.

A further embodiment provides that for calculating the corrected Zernike polynomials, the corresponding Zernike polynomials are adapted for higher order aberrations by a translation in opposite direction to the offset vector. In other words, the Zernike polynomials are shifted in opposite direction of the offset vector for the correction. Thus, a spherical aberration and/or coma and/or trefoil can in particular be corrected by the offset vector, whereby spherical and cylindrical refraction components arise, which can be taken into account in the subjective refraction.

In a further embodiment a difference of the Zernike polynomials to the corrected Zernike polynomials is examined for exceeding a tolerance threshold, wherein a warning message is provided if the difference is above the tolerance threshold. This means that if the offset vector is too large and thereby an adaptation of the Zernike polynomials exceeds a tolerance threshold, then, a warning message is to be output. For example, a warning message can be output if a difference of transformed Zernike polynomials with respect to a subjective refraction is above a tolerance threshold. Analogously or alternatively thereto, the warning message can be output if differences between the Zernike polynomials derived from the subjective refraction and the measured Zernike polynomials, which are both related to the pupil center, are above a (further) tolerance threshold.

Furthermore, a method for controlling a treatment apparatus is provided. Therein, the method includes the method steps of at least one embodiment of a method as it was previously described. Furthermore, the method for controlling the treatment apparatus also includes the step of transferring the provided control data to at least one ophthalmological laser of the treatment apparatus. Preferably, the treatment apparatus or the laser can subsequently be controlled with the control data for correcting a visual disorder.

The respective method can include at least one additional step, which is executed if and only if an application case or an application situation occurs, which has not been explicitly described here. For example, the step can include the output of an error message and/or the output of a request for inputting a user feedback. Additionally or alternatively, it can be provided that a default setting and/or a predetermined initial state are adjusted.

A further aspect of the invention relates to a control device, which is formed to perform the steps of at least one embodiment of one or both of the previously described methods. Thereto, the control device can comprise a computing unit for electronic data processing such as for example a processor. The computing unit can include at least one microcontroller and/or at least one microprocessor. The computing unit can be configured as an integrated circuit and/or microchip. Furthermore, the control device can include an (electronic) data memory or a storage unit. A program code can be stored on the data memory, by which the steps of the respective embodiment of the respective method are encoded. The program code can include the control data for the respective laser. The program code can be executed by means of the computing unit, whereby the control device is caused to execute the respective embodiment. The control device can be formed as a control chip or control unit. The control device can for example be encompassed by a computer or computer cluster.

A further aspect of the invention relates to a treatment apparatus with at least one eye surgical or ophthalmological laser and a control device, which is formed to perform the steps of at least one embodiment of one or both of the previously described methods. The respective laser can be formed to at least partially separate a predefined corneal volume with predefined interfaces of a human or animal eye by means of optical breakthrough, in particular at least partially separate it by means of photodisruption and/or to ablate corneal layers by means of (photo)ablation and/or to effect a laser-induced refractive index change in the cornea and/or the eye lens.

In a further advantageous configuration of the treatment apparatus according to the invention, the laser can be suitable to emit laser pulses in a wavelength range between 300 nm and 1400 nm, preferably between 900 nm and 1200 nm, at a respective pulse duration between 1 fs and 1 ns, preferably between 10 fs and 10 ps, and a repetition frequency of greater than 10 kilohertz (kHz), preferably between 100 kHz and 100 megahertz (MHz). The use of such lasers in the method according to the invention additionally has the advantage that the irradiation of the cornea does not have to be effected in a wavelength range below 300 nm. This range is subsumed by the term “deep ultraviolet” in the laser technology. Thereby, it is advantageously avoided that an unintended damage to the cornea is effected by these very short-wavelength and high-energy beams. Photodisruptive and/or ablative lasers of the type used here usually input pulsed laser radiation with a pulse duration between 1 fs and 1 ns into the corneal tissue. Thereby, the power density of the respective laser pulse required for the optical breakthrough can be spatially narrowly limited such that a high incision accuracy is allowed in the generation of the interfaces. In particular, the range between 700 nm and 780 nm can also be selected as the wavelength range.

In further advantageous configurations of the treatment apparatus according to the invention, the control device can comprise at least one storage device for at least temporary storage of at least one control dataset, wherein the control dataset or datasets include(s) control data for positioning and/or for focusing individual laser pulses in the cornea; and can comprise at least one beam device for beam guidance and/or beam shaping and/or beam deflection and/or beam focusing of a laser beam of the laser.

A further aspect of the invention relates to a computer program. The computer program includes commands, which for example form a program code. The program code can include at least one control dataset with the respective control data for the respective laser. Upon execution of the program code by means of a computer or a computer cluster, it is caused to execute the previously described method or at least one embodiment thereof.

A further aspect of the invention relates to a computer-readable medium (storage medium), on which the above mentioned computer program and the commands thereof, respectively, are stored. For executing the computer program, a computer or a computer cluster can access the computer-readable medium and read out the content thereof. The storage medium is for example formed as a data memory, in particular at least partially as a volatile or a non-volatile data memory. A non-volatile data memory can be a flash memory and/or an SSD (solid state drive) and/or a hard disk. A volatile data memory can be a RAM (random access memory). For example, the commands can be present as a source code of a programming language and/or as assembler and/or as a binary code.

Further features and advantages of one of the described aspects of the invention can result from the embodiments of another one of the aspects of the invention. Thus, the features of the embodiments of the invention can be present in any combination with each other if they have not been explicitly described as mutually exclusive.

### BRIEF DESCRIPTION OF THE DRAWINGS

In the following, additional features and advantages of the invention are described in the form of advantageous execution examples based on the figure(s). The features or feature combinations of the execution examples described in the following can be present in any combination with each other and/or the features of the embodiments. This means, the features of the execution examples can supplement and/or replace the features of the embodiments and vice versa. Thus, configurations are also to be regarded as encompassed and disclosed by the invention, which are not explicitly shown or explained in the figures, but arise from and can be generated by separated feature combinations from the execution examples and/or embodiments. Thus, configurations are also to be regarded as disclosed, which do not comprise all of the features of an originally formulated claim or extend beyond or deviate from the feature combinations set forth in the relations of the claims. To the execution examples, there shows:

FIG. 1 depicts a schematic representation of a treatment apparatus according to an exemplary embodiment.

FIG. 2 depicts a schematic method diagram according to an exemplary embodiment.

In the figures, identical or functionally identical elements are provided with the same reference characters.

### DETAILED DESCRIPTION

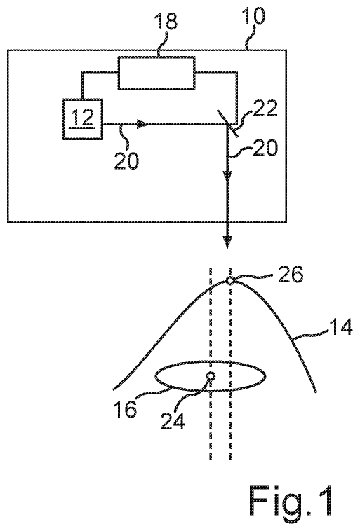

The FIG. 1 shows a schematic representation of a treatment apparatus 10 with an eye surgical laser 12 for the correction of a cornea 14, wherein a correction profile, in particular a volume body or a lenticule (not shown), can for example be defined by control data, which can be separated from the cornea 14 by means of photodisruption and/or ablation. For separating the lenticule, interfaces can for example be preset in the control data, on which a cavitation bubble path for separating the lenticule from the cornea 14 can be generated.

One recognizes that a control device 18 for the laser 12 can be formed besides the laser 12, such that it can emit pulsed laser pulses for example in a predefined pattern for generating the correction profile or the interfaces. Alternatively, the control device 18 can be a control device 18 external with respect to the treatment apparatus 10.

Furthermore, the FIG. 1 shows that the laser beam 20 generated by the laser 12 is deflected towards the cornea 14 by means of a beam device 22, namely a beam deflection device such as for example a rotation scanner. The beam deflection device 22 is also controlled by the control device 18 to generate the correction profile or the interfaces.

Preferably, the illustrated laser 12 can be a photodisruptive and/or ablative laser, which is formed to emit laser pulses in a wavelength range between 300 nm and 1400 nm, preferably between 700 nm and 1200 nm, at a respective pulse duration between 1 fs and 1 ns, preferably between 10 fs and 10 ps, and a repetition frequency of greater than 10 kHz, preferably between 100 kHz and 100 MHz. In addition, the control device 18 optionally comprises a storage device (not illustrated) for at least temporary storage of at least one control dataset, wherein the control dataset or datasets include(s) control data for positioning and/or for focusing individual laser pulses in the cornea. The position data and/or the focusing data of the individual laser pulses, that is the correction profile of the lenticule to be separated, are generated based on predetermined control data, in particular from previously measured visual disorder correction data, in particular a previously measured topography and/or pachymetry and/or the morphology and/or an aberrometry, in particular wavefront data, of the cornea.

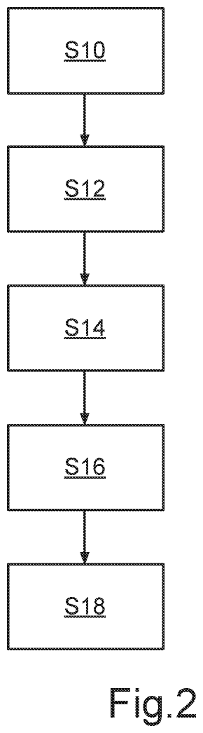

In case of a deformed or asymmetric cornea 14, as it is schematically illustrated in FIG. 1, it can occur that the pupil center 24 of the pupil 16 does not coincide with a visual axis, which can be related to a corneal vertex 26. Thus, in planning the correction profile, according to used method for planning, an offset between reference centers, in particular the pupil center 24 and the corneal vertex 26, is present. If the correction profile is for example planned based on wavefront measurements by means of an aberrometer, the pupil center 24 serves as the reference center, since the wavefronts are measured through the pupil 16. In contrast, if the correction profile is for example planned based on a topography, the corneal vertex 26 usually serves as the reference center, since it specifies the highest point of the topography. In order to consider this discrepancy in planning the correction profile, the control device 18 can be formed to perform the method schematically illustrated in FIG. 2 for providing control data for the ophthalmological laser 12 of the treatment apparatus 10, to correct, in particular higher order, aberrations, which can occur by an offset of the reference centers.

In a step S10, visual disorder correction data for correcting the cornea 14 of the eye can be ascertained. Hereto, different diagnostic methods can be applied, for example wavefront measurements by means of an aberrometer, topology measurements and/or visual disorder data can be determined based on a subjective refraction or glasses correction, which can be measured by means of a phoropter.

In a step S12, Zernike polynomials, which are adjusted based on a first reference center, can then be determined from the ascertained visual disorder correction data. Herein, there is for example the possibility that the Zernike polynomials can be directly ascertained from an aberrometer measurement and the wavefronts obtained therefrom, wherein the pupil center 24 serves as the first reference center for this measurement. Alternatively, at least second order Zernike polynomials can for example be ascertained from the subjective refraction of the glasses correction, which is provided by means of the phoropter measurement, wherein the visual axis or the corneal vertex 26 serves as the first reference center in this case.

In a step S14, an offset vector from the first reference center, for example the pupil center 24, to a second reference center of the eye can then be ascertained, wherein the second reference center is preferably the projection of the corneal vertex 26 to the plane of the pupil center 24 in this example. Alternatively, an offset vector to further preset reference centers, for example a visual axis, an optical axis and/or an achromatic axis, can also be determined. Accordingly, one thus obtains a difference between optical systems of the eye, which is to be taken into account for correcting the higher order aberrations.

Accordingly, it can be provided in a step S16 that the previously ascertained Zernike polynomials are corrected by means of the offset vector, in particular by a translation and/or rotation, to adapt higher order aberrations for the correspondingly other reference center. In particular, the Zernike polynomials for defocus, astigmatism, coma, spherical aberration and/or trefoil can be adapted by means of the offset vector.

Below, two preferred cases of application for the previously shown steps are described. In the first case of application, the visual disorder correction data can be wavefront measurements, which have the pupil center 24 as the first reference center, since they can only be measured through the pupil 16. However, since the pupil center 24 does not correspond to the visual axis, which can preferably be adjusted to the corneal vertex 26, and which serves as the second reference center in this example, the Zernike polynomials, which originate from the wavefront measurement, are shifted to the actual visual range. By means of the offset vector, which specifies a distance of the reference centers, the Zernike polynomials, in particular the Zernike polynomials for higher order aberrations, can be adapted or shifted such that they describe the aberrations from the view of the visual axis.

A further preferred application can be that the visual disorder correction data originates from a subjective refraction, which is performed by means of a phoropter measurement. Herein, the refraction and in particular aberrations, which can be described by second order Zernike polynomials, can be determined by measurements based on a perception of the patient, wherein the perception of the patient corresponds to the visual axis. In other words, the visual axis or the corneal vertex 26 can serve as the first reference center for the visual disorder correction data in this case, wherein the Zernike polynomials, in particular of the second order, ascertained from the visual disorder correction data, are thus also adjusted based on the visual axis. Herein, the development of the aberrations, which can in particular include astigmatism and defocus, can occur due to an offset of the visual axis (corneal vertex 26) to a second reference center, in particular to the pupil center 24, since the optical systems of the eye are not coaxially situated. Accordingly, this difference can be compensated for by adaptation of the Zernike polynomials by ascertaining the offset vector.

Furthermore, it can preferably be provided that a difference between the original Zernike polynomials and the Zernike polynomials corrected by the offset vector is also examined to the effect if it exceeds a tolerance threshold. If this is the case, a warning message can be output to indicate that the difference is very large. Thus, an attending physician can for example better decide whether or not these corrections are to be performed.

Finally, control data for the treatment apparatus 10 can be provided in a step S18, which is ascertained by the corrected Zernike polynomials and in which the higher order aberrations can be compensated for due to the corrected Zernike polynomials.

1. A method for providing control data for an ophthalmological laser of a treatment apparatus, wherein the method comprises the following steps performed by a control device:
ascertaining visual disorder correction data for correcting a cornea of an eye;
determining Zernike polynomials from the ascertained visual disorder correction data, which are adjusted based on a first reference center of the eye;
ascertaining an offset vector from the first reference center to a further second reference center of the eye;
calculating corrected Zernike polynomials, in which higher order aberrations are calculated by means of an adaptation of the corresponding Zernike polynomials by the offset vector;
providing the control data for the treatment apparatus, wherein the control data is generated by means of the corrected Zernike polynomials.

2. The method according to claim 1, wherein at least the Zernike polynomials for defocus, astigmatism, coma, trefoil and/or spherical aberration are adapted by means of the offset vector.

3. The method according to claim 1, wherein the first reference center is a pupil center of the eye and the Zernike polynomials are corrected towards the second reference center, in particular towards a corneal vertex, by the offset vector.

4. The method according to claim 1, wherein the first reference center is a visual axis of the eye, in particular a corneal vertex, and Zernike polynomials at least of the second order are determined based on a predetermined subjective refraction of a glasses correction, which is in particular ascertained by means of a phoropter measurement, wherein these Zernike polynomials are corrected by the offset vector.

5. The method according to claim 1, wherein for calculating the corrected Zernike polynomials, the corresponding Zernike polynomials are adapted for higher order aberrations by a translation in opposite direction to the offset vector.

6. The method according to claim 1, wherein a difference of the Zernike polynomials to the corrected Zernike polynomials is examined for exceeding a tolerance threshold, wherein a warning message is provided if the difference is above the tolerance threshold.

7. A method for controlling a treatment apparatus, wherein the method comprises the following steps:
the method steps according to claim 1, and
transferring the provided control data to a respective eye surgical laser of the treatment apparatus.

8. A control device, which is configured to perform a respective method according to claim 1.

9. A treatment apparatus with at least one eye surgical laser for separation of a corneal volume with predefined interfaces of a human or animal eye by means of optical breakthrough, in particular by means of photodisruption and/or photoablation, and at least one control device according to claim 8.

10. A non-transitory computer-readable medium, configured for storing a computer program, the computer program including commands which cause a treatment apparatus to execute a method according to claim 1.

11. (canceled)
